(19) United States
(12) Patent Application Publication
(10) Pub. No.: US 2024/0197149 A1
(43) Pub. Date: Jun. 20, 2024

(54) ENDOSCOPE VALVE DEVICES, SYSTEMS, AND METHODS

(71) Applicant: BOSTON SCIENTIFIC SCIMED, INC., MAPLE GROVE, MN (US)

(72) Inventor: Nathan Thomas Cummings, Worcester, MA (US)

(73) Assignee: BOSTON SCIENTIFIC SCIMED, INC., MAPLE GROVE, MN (US)

(21) Appl. No.: 18/541,313

(22) Filed: Dec. 15, 2023

Related U.S. Application Data
(60) Provisional application No. 63/433,063, filed on Dec. 16, 2022.

(51) Int. Cl. A61B 1/00; A61B 1/015

(52) U.S. Cl. CPC A61B 1/00068; A61B 1/015

(57) ABSTRACT

A valve assembly with a valve shaft shiftable within a valve well from an off position blocking fluid communication between ports in the valve well, and an on position allowing fluid communication between such ports. The on position may be proximal to the off position. The length of the valve shaft is selected simply to block access to one of the ports of the valve well when in the off position, and thus may be shorter than prior valve shafts. The valve shaft is along a distal end of an actuatable member of the valve assembly, and a user-engagement element may be provided along the proximal end of the actuatable member. The user-engagement element may be movable towards the valve shaft to move the valve shaft from the off position to the on position.

### CROSS-REFERENCE TO RELATED APPLICATIONS

The present application claims the benefit of priority under 35 U.S.C. § 119 to U.S. Provisional Application No. 63/433,063, filed Dec. 16, 2022, all of the disclosure of which is hereby incorporated herein by reference in its entirety for all purposes. Any and all priority claims identified in the Application Data Sheet, or any correction thereto, are hereby incorporated by reference under 37 C.F.R. § 1.57.

### FIELD

The present disclosure relates generally to devices (including, without limitation, components and assemblies), systems, and methods for controlling flow of materials through a valve. In particular, the present disclosure relates to devices, systems, and methods for controlling flow of materials through a valve assembly usable in a medical device such as an endoscope.

### BACKGROUND

Various devices with valve assemblies are known in the art for use during various medical procedures. For instance, materials may be supplied to an anatomical site (e.g., fluid may be supplied, such as for irrigation), and/or suctioned from the anatomical site (e.g., fluid or biological materials may be withdrawn from an anatomical site) during a medical procedure. A valve assembly may be used to control flow of such materials. An endoscope is a common medical device used to introduce or remove substances with respect to an anatomical site, and thus typically includes a valve assembly. Endoscopes typically have an insertion tube with a working channel via which substances (e.g., fluids such as gas or liquids) or devices or instruments or tools may be introduced, or substances may be removed or suctioned out. To control flow of substances through an endoscope, a fluid source, and/or a suction pump/vacuum source is fluidly coupled with the endoscope handle and the insertion tube via a valve assembly. The valve assembly typically has a valve well and a valve shaft shiftable within the valve well between an off position in which the valve assembly is in an off/closed configuration, and an on position in which the valve assembly is in an on/open configuration. In the off configuration, the valve assembly blocks fluid communication between the fluid source/suction source and the insertion tube of the endoscope. When the valve assembly is shifted into an on configuration (typically by being depressed with respect to the handle), fluid communication between the fluid source/suction source and the working channel of the endoscope is established to supply fluid and/or to apply suction/negative pressure to the insertion tube of the endoscope. Proper sealing of the ports, channels, lumens, etc., associated with such valve assemblies is important. However, seals which provide sealing interference can also create a large drag force which may affect operation of the valve shaft as it is shifted, often repeatedly, within the valve well. There remains a need for improvements to endoscope valves, such as suction valves and arrangements and operations thereof.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

An actuatable member is formed in accordance with various principles of the present disclosure for a valve assembly of a medical instrument. The valve assembly may include a valve well having a proximal end and a distal end, and defining a valve well passage extending therebetween and along an actuation axis. The valve shaft has an actuatable member having a proximal end and a distal end, the actuatable member extending through the valve well passage and shiftable therein, along the actuation axis, between a first position and a second position. The actuatable member includes a user-engagement element along the proximal end thereof, and a shaft along the distal end thereof, movable with respect to the user-engagement element. Distal movement of the user-engagement element toward the distal end of the valve well causes proximal movement of the valve shaft toward the proximal end of the valve well.

Optionally, a biasing assembly couples the user-engagement element and the valve shaft. Optionally, the biasing assembly is configured to shift between an extended configuration, in which the user-engagement element and the valve shaft are spaced apart from each other a first distance, and a contracted configuration, in which the user-engagement element and the valve shaft are spaced apart from each other a second distance shorter than the first distance. Optionally, the biasing assembly includes one or more linkages configured to shift between an extended configuration, in which the user-engagement element and the valve shaft are spaced apart from each other a first distance, and a contracted configuration, in which the user-engagement element and the valve shaft are spaced apart from each other a second distance shorter than the first distance. Optionally the biasing assembly comprises a linkage assembly comprising a plurality of linkages pivotable coupled together between the user-engagement element and the valve shaft. In some embodiments, the plurality of linkages includes proximal linkages having proximal ends and distal ends, and distal linkages having proximal ends and distal ends, with the proximal ends of the proximal linkages coupled to the user-engagement element, the distal ends of the distal linkages are coupled to the valve shaft, and the distal ends of the proximal linkages and the proximal ends of the distal linkages are pivotably coupled with respect to one another. Optionally, a guide path positioned with respect to the linkage assembly guides a portion of the one or more linkages to move the linkages between the extended configuration and the compressed configuration. Optionally, the distal ends of the proximal linkages and the proximal ends of the distal linkages are guided with respect to the guide path to move along a guide axis transverse to the actuation axis to shift the linkage assembly between the expanded configuration and the contracted configuration.

In some embodiments, a linkage assembly which couples the user-engagement element and the valve shaft, the linkage assembly having at least one linkage having a proximal end coupled to the user-engagement element and a distal end coupled to the valve shaft. Optionally, the at least one linkage is formed of a resilient material; and distal movement of the user-engagement element toward the distal end of the valve well causes the at least one linkage to flex and to shift the valve shaft toward the proximal end of the valve well. Optionally, a guide path is positioned with respect to the linkage assembly to guide a portion of the at least one linkage between a medial position with respect to the valve assembly and a laterally outward position with respect to the valve assembly laterally away from the actuation axis to aid in flexing of the at least one linkage into the contracted configuration.

Optionally, the user-engagement element is biased away from the distal end of the valve well.

In some aspects, an axially-extending port is in fluid communication with the valve well passage along the actuation axis; a transversely-extending port is in fluid communication with the valve well passage in a direction transverse to the actuation axis; and distal movement of the user-engagement element shifts the valve shaft from a position blocking fluid communication between the axially-extending port and the transversely-extending port via the valve well passage, to a position allowing fluid communication between the axially-extending port and the transversely-extending port via the valve well passage.

In accordance with various further principles of the present disclosure, an actuatable member is formed for a valve assembly of a medical instrument with a valve shaft extendable within a valve well passage between an off position, in which the valve shaft blocks fluid communication between an axially-extending port and a transversely-extending port via the valve well passage, and an on position, in which the valve shaft is moved out of the flow path between the axially-extending port and the transversely-extending port via the valve well passage to allow fluid communication therebetween.

Optionally, a user-engagement element is coupled to the valve shaft and is movable distally to shift the valve shaft from the valve shaft off position to the valve shaft on position. Optionally, the valve assembly includes an assembly coupling the user-engagement element and the valve shaft. In some aspects, the biasing assembly includes a linkage assembly having one or more linkages configured to shift between an extended configuration in which the user-engagement element and the valve shaft are spaced apart from each other a first distance, and a contracted configuration in which the user-engagement element and the valve shaft are spaced apart from each other a second distance shorter than the first distance. Optionally, the valve assembly includes a guide path positioned with respect to the linkage assembly to guide a portion of the one or more linkages to move the linkages between the extended configuration and the contracted configuration.

In some aspects, when the valve shaft is in the off position, the valve shaft is positioned to block a flow path between the axially-extending port and the transversely-extending port; and when the valve shaft is in the on position, the valve shaft is positioned proximal to the transversely-extending port and out of the flow path between the axially-extending port and the transversely-extending port.

A valve shaft and/or valve assembly formed in accordance with one or more of the various principles of the present disclosure may be operatively associated with a control handle of an endoscope.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers with a ′ added at the end, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of an endoscope with one or more valves formed in accordance with aspects of the present disclosure.

FIG. 2A illustrates a cross-sectional view, along line I-I of FIG. 1, of an example of an embodiment of a valve assembly which may be provided in an endoscope as illustrated in FIG. 1, the valve assembly being illustrated in an off or closed configuration.

FIG. 2B illustrates a cross-sectional view similar to that of FIG. 2A, but with the valve assembly in an on or open configuration.

FIG. 3A illustrates a schematic cross-sectional view of a valve assembly such as illustrated in FIG. 2A, but also illustrating an example of an embodiment of a biasing assembly.

FIG. 3B illustrates a schematic cross-sectional view of a valve assembly such as illustrated in FIG. 2B, but also illustrating an example of an embodiment of a biasing assembly.

FIG. 4A illustrates a schematic cross-sectional view of a valve assembly such as illustrated in FIG. 2A, but also illustrating another example of an embodiment of a biasing assembly.

FIG. 4B illustrates a schematic cross-sectional view of a valve assembly such as illustrated in FIG. 2B, but also illustrating another example of an embodiment of a biasing assembly.

FIG. 5A illustrates a schematic cross-sectional view of a valve assembly such as illustrated in FIG. 2A, but also illustrating another example of an embodiment of a biasing assembly.

FIG. 5B illustrates a schematic cross-sectional view of a valve assembly such as illustrated in FIG. 2B, but also illustrating another example of an embodiment of a biasing assembly.

FIG. 6A illustrates a cross-sectional view, along line I-I of FIG. 1, of another example of an embodiment of a valve assembly which may be provided in an endoscope as illustrated in FIG. 1, the valve assembly being illustrated in an off or closed configuration.

FIG. 6B illustrates a cross-sectional view similar to that of FIG. 6A, but with the valve assembly in an on or open configuration.

FIG. 7A illustrates a cross-sectional view, along line I-I of FIG. 1, of another example of an embodiment of a valve assembly which may be provided in an endoscope as illustrated in FIG. 1, the valve assembly being illustrated in an off or closed configuration.

FIG. 7B illustrates a cross-sectional view similar to that of FIG. 7A, but with the valve assembly in an on or open configuration.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user n (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

Various medical devices include valve assemblies to regulate or control fluid delivery (irrigation) or fluid suction (aspiration) with respect to an anatomical site. Although the present disclosure describes suction valves, it will be appreciated that the principles of the present disclosure need not be so limited.

A suction valve assembly of a medical device is arranged to apply suction from a suction source to an anatomical site, such as via a flexible tubular element which is configured and positionable with respect to the anatomical site. The medical device may be an endoscope, and the flexible tubular element may be an insertion tube of the endoscope, however the present disclosure need not be so limited. The suction source may be a pump or other mechanism creating a vacuum to be applied to the anatomical site via the flexible tubular element, the present disclosure not being limited in this regard. In the off configuration of the valve assembly, fluid communication between the suction source and the flexible tubular element is cut off or blocked so that suction is not applied to the anatomical site, and the valve may be considered to be in a closed configuration. In the on configuration of the valve assembly, the suction source is fluidly coupled with the flexible tubular element, such as to aspirate an anatomical site, and the valve may be considered to be in an open configuration.

Valve assemblies of medical devices may be mounted with respect to a control handle, and typically include a valve well and an actuatable member movable with respect to the valve well to shift the valve assembly between the off configuration and the on configuration. The valve well is formed in, or formed and positioned within, the control handle. The actuatable member may include a user-engagement element and a valve shaft. The valve shaft is movable, along an actuation axis, within a valve-well channel extending through the valve well. Various valve assemblies have different arrangements of ports and flow paths placing a fluid source, such as a suction source, in and out of fluid communication with and anatomical site, such as via a flexible tubular element. For instance, in some valve assemblies, the suction source is fluidly coupled with a suction source port in the valve well which extends generally transverse to the actuation axis of the valve shaft. In such valve assemblies, the flexible tubular element is typically fluidly coupled with a suction application port in the valve well which is generally axially aligned with the actuation axis of the valve shaft. In other valve assemblies, the suction source is fluidly coupled with a suction source port in the valve well which is generally axially aligned with the actuation axis of the valve shaft. In such valve assemblies, the flexible tubular element is typically fluidly coupled with a suction application port in the valve well which extends generally transverse to the actuation axis of the valve shaft. In both such valve assemblies, the flexible tubular element extends from the control handle to an anatomical site within a patient to apply suction. Principles of the present disclosure may be applied to either configuration of a valve assembly.

In accordance with various principles of the present disclosure, a simplified valve shaft is provided within the valve well of a valve assembly. The valve shaft formed in accordance with various principles of the present disclosure is generally shorter than prior art valve shafts and does not include holes, suction passages, ports, etc., communicating the suction source port and the suction application port of the valve well in the different positions of the valve shaft with respect to the valve well. Instead, the simplified valve shaft of the present disclosure is sized, shaped, configured, and/or dimensioned simply to block the transversely-extending port in the valve well when the valve shaft is in an off position, thereby placing the valve assembly in an off or closed configuration. The simplified valve shaft of the present disclosure is selectively movable out of the position blocking the transversely-extending port in the valve well to place the valve assembly in an on or open configuration. At least a portion of the circumferential surface positioned adjacent the transversely-extending port is configured to seal fluid communication with the transversely-extending port of the valve well when the valve shaft is in the off position. Optionally, the distal surface of the valve shaft does not include flow passages therethrough and/or is configured to seal fluid communication with the axially-extending port of the valve well. The simplified valve shaft may be formed of a material capable of assuring a tight seal with respect to the transversely-extending port of the valve well. As may be appreciated, if the valve shaft need only seal the transversely-extending port in the valve well, the length of the valve shaft need only be sufficient to seal such port, and therefore need only be sufficiently larger than the diameter of the transversely-extending port to seal the port. Typically, the transversely-extending port meets the valve well at an angle, so that the port at the intersection may have a generally elliptical cross-sectional shape, and therefore may be generally larger than the passage extending therefrom. For instance, the intersection of the transversely-extending port with the valve well may define an ellipse with a major axis of approximately 6 mm, whereas, the diameter of the typically circular cross-section of the passage extending therefrom (away from the valve well) may be 4 mm. As such, the length of a valve sized, shaped, configured, and/or dimensioned to seal the transversely-extending port, as may be appreciated, is generally significantly shorter than the length of prior art valve shafts for valve assemblies.

As noted above, the valve shaft may be considered to be part of an actuatable member having a user-engagement element (e.g., push button, cap, etc.) for engagement by a user's hand or finger to operate the actuatable member. Further in accordance with various principles of the present disclosure, instead of movement of the user-engagement element causing movement of the valve shaft in the same direction to shift the valve assembly from one position to another position, movement of the user-engagement element causes movement of the valve shaft in an opposite direction. More particularly, movement of the user-engagement element of an actuatable member in a distal direction (towards the valve well) causes proximal movement of the valve shaft towards/closer to the user-engagement element. Conversely, movement of the user-engagement element of an actuatable member in a proximal direction (away from the valve well) causes distal movement of the valve shaft away from the user-engagement element. Such configuration of an actuatable member facilitates movement of a valve shaft formed in accordance with various principles of the present disclosure from a position blocking fluid communication between the valve passage in the valve well and the transversely-extending port of the valve well (and thus also with the axially-extending port of the valve well), to a position out of the flow path between the transversely-extending port and the axially-extending port of the valve well to allow unimpeded fluid flow therebetween. In accordance with various principles of the present disclosure, a biasing assembly, system, mechanism, etc. (such terms may be used interchangeably herein without intent to limit), may be provided between the user-engagement element and the valve shaft to maintain the valve assembly in a closed, off configuration. A user may actuate (e.g., push, depress, apply an actuation force to, etc.) the user-engagement element to shift the valve assembly to an open, on configuration. Once the user stops actuating the user-engagement element, the biasing assembly may return the valve assembly to the closed, off configuration by moving apart the user-engagement element and the valve shaft.

For the sake of convenience, and without intent to limit, reference is made herein to a valve assembly for a suction valve of an endoscope. The flexible tubular element of the endoscope is referenced herein as an insertion tube, and is generally positionable within a patient, such as within an organ, body lumen/passageway, cavity, etc. (reference being made herein to any or other such anatomical sites without intent to limit). The insertion tube defines one or more lumens therethrough configured for passage of materials, instruments, tools, devices, etc., through the working channel to an anatomical site. For instance, the lumens may include a suction lumen, an irrigation lumen, a working channel, and/or a visualization lumen (e.g., for a light guide, optic fiber, camera element, etc.).

Various embodiments of valve devices (including, without limitation, components and assemblies), systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers followed by a ′ and/or increased by 100, and redundant description is generally omitted for the sake of brevity. Moreover, certain features in one embodiment may be used across different embodiments and are not necessarily individually labeled when appearing in different embodiments.

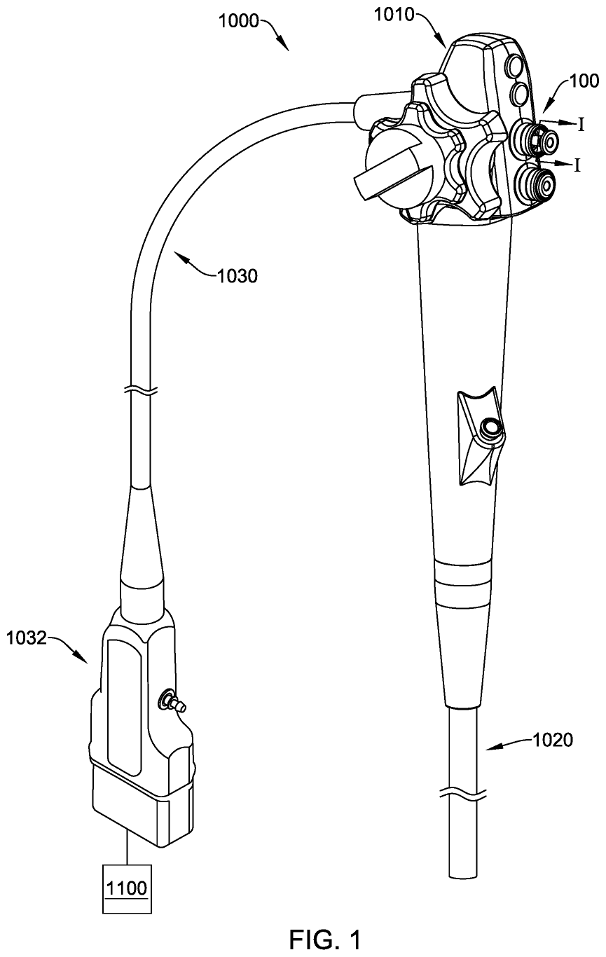

Turning now to the drawings, an example of an embodiment of a valve assembly 100 formed in accordance with various principles of the present disclosure is illustrated in FIG. 1 as provided in an example of an embodiment of an endoscope 1000. It will be appreciated that the endoscope 1000 is an example of an embodiment in which principles of the present disclosure may be applied, and that various principles of the present disclosure are applicable to other medical instruments to control fluid flow with respect thereto, the details of which are not critical to the present disclosure. Moreover, although reference is made to a suction valve, it will be appreciated that the disclosed principles and embodiments are applicable to other valves, such as fluid supply/irrigation valves.

The illustrated example of an embodiment of a valve assembly 100 is mounted with respect to a control handle 1010 of the endoscope 1000 to regulate the flow of materials (e.g., fluid) between the insertion tube 1020 of the endoscope 1000 and a suction source 1100. The endoscope 1000 has a connector cord 1030 extending to a scope connector 1032 with which the endoscope 1000 (and valve assembly 100) may be fluidly coupled with the suction source 1100. The connector cord 1030 may be alternatively referenced herein as an umbilical cord, umbilicus, universal cord, etc., without intent to limit. The scope connector 1032 may also couple the endoscope 1000, via the connector cord 1030, with a variety of components, devices, etc., such as a fluid source (to supply air, carbon dioxide, water, saline, or other gases or liquids), electrical connections, light sources, visualization elements (e.g., optic fibers, cameras, etc.), or other components, devices, etc., usable with the endoscope 1000. The insertion tube 1020 has a fluid lumen extending therethrough to a distal end which is positionable (insertable, navigable, etc.) with respect to an anatomical site (e.g., within a patient). Similarly, the connector cord 1030 has a fluid lumen extending therethrough to fluidly couple the suction source 1100 (e.g., via the scope connector 1032) with the control handle 1010. The fluid lumens through the insertion tube 1020 and the connector cord 1030, and the distal end of the insertion tube 1020, may be well-known features formed in a manner known to those of ordinary skill in the art and are not illustrated to simplify the drawings by eliminating details in the illustration of the endoscope 1000 in FIG. 1 which are not necessary for understanding the present disclosure.

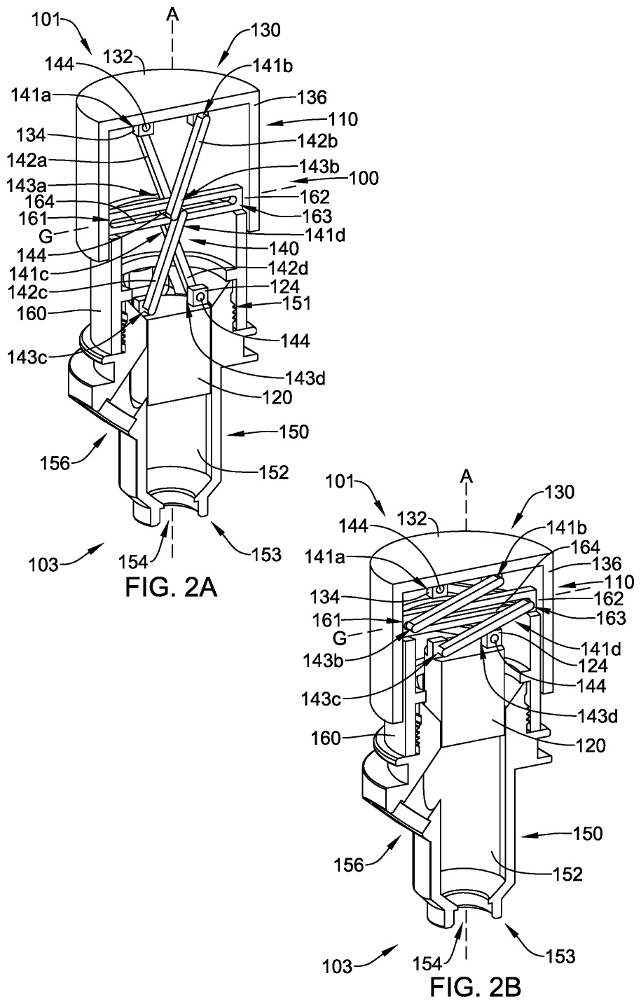

An example of an embodiment of a valve assembly 100 formed in accordance with various principles of the present disclosure is illustrated in FIG. 2A and FIG. 2B, in isolation from an endoscope (such as the endoscope 1000 illustrated in FIG. 1). In the illustrated example of an embodiment, the valve assembly 100 includes an actuatable member 110 extending from a proximal end 101 of the valve assembly 100. The distal end 103 of the valve assembly 100 is typically positioned or housed within a control handle or other suitable housing. The actuatable member 110 includes a valve shaft 120 movable within the valve assembly 100, and a user-engagement element 130 accessible (outside any housing the valve shaft 120 extends within) by a user to engage the user-engagement element 130. The valve shaft 120 and the user-engagement element 130 are operatively coupled via a linkage assembly 140 which transmits motion of the user-engagement element 130 to the valve shaft 120, as will be described in further detail below. The valve shaft 120 is actuated by engagement of the user-engagement element 130 by a user (e.g., along a user-engagement surface 132) and application of an actuation force thereto (e.g., along an actuation axis A) to move the actuatable member 110, and thus the valve shaft 120, between an off (closed) position (as illustrated in FIG. 2A) and an on (open) position (as illustrated in FIG. 2B) within a valve well 150 of the valve assembly 100. When the valve shaft 120 is in the off (closed) position, the valve assembly 100 is in an off configuration and does not apply suction to a suction application device fluidly coupled with the valve assembly 100 (e.g., an insertion tube of an endoscope). When the valve shaft 120 is in the on (open) position, the valve assembly 100 is in an on configuration and may apply suction to a suction application device. The actuatable member 110 may be mounted with respect to the valve well 150 via a collar 160 which is coupled to the valve well 150, such as with snap-fit features such as known to those of ordinary skill in the art. The collar 160 may also serve to guide movement of the actuatable member 110 to shift the valve shaft 120 between its off and on positions, as described in further detail below.

In the example of an embodiment of a valve assembly 100 illustrated in FIG. 2A and FIG. 2B, the valve shaft 120 is positioned within a passage 152 within the valve well 150 extending along an actuation axis A. The valve well 150 has an axially-extending port 154 allowing fluid flow generally axially with respect to the valve well passage 152, and a transversely-extending port 156 allowing fluid flow generally transverse to the valve well passage 152. The valve shaft 120 is movable/shiftable with respect to the valve well 150 and within the valve well passage 152 along the actuation axis A. In its off position, such as illustrated in FIG. 2A, the valve shaft 120 blocks fluid flow between the axially-extending port 154 and the transversely-extending port 156 of the valve well 150. As such, a fluid source in fluid communication with one of the ports 154, 156 is blocked by the valve shaft 120 from fluid communication with the other of the ports 154, 156. In its on position, such as illustrated in FIG. 2B, the valve shaft 120 is shifted out of a position blocking fluid communication between the axially-extending port 154 and the transversely-extending port 156. The valve assembly 100 is thereby shifted into an on configuration in which fluid may be communicated between a fluid source and a fluid application device. For instance, the fluid source may be a suction source (e.g., a vacuum pump), and the fluid application device an insertion tube of an endoscope with a working channel in fluid communication with the valve well 150, and in fluid communication with the suction source via the valve well passage 152 to apply suction to a target site within a patient.

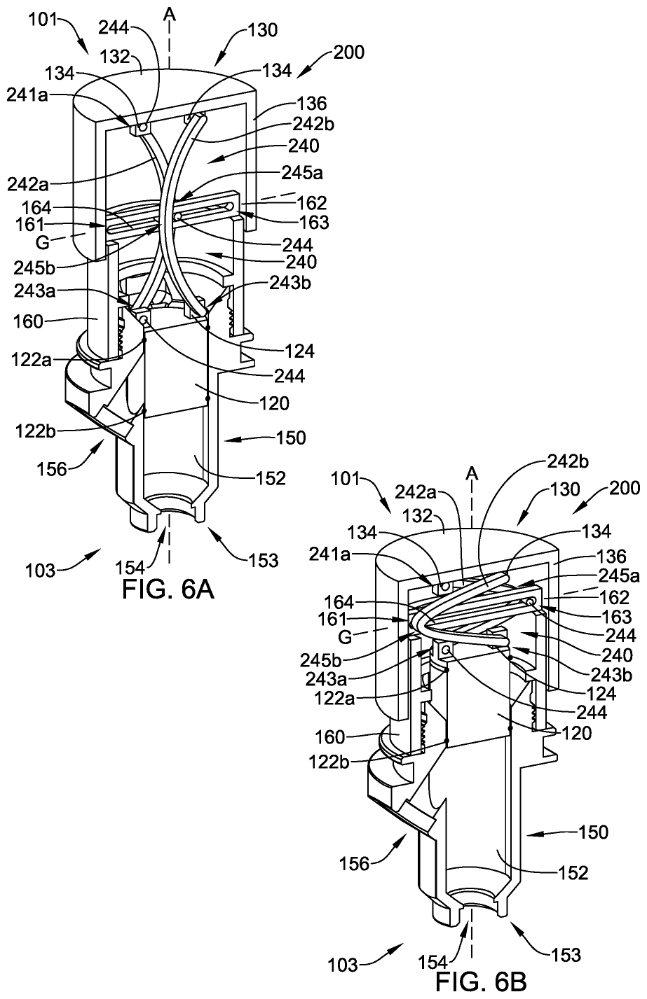

As may be appreciated, the valve shaft 120 need not extend the full length of the valve well passage 152 along the actuation axis A. For instance, the valve shaft 120 may have a length slightly greater than the diameter of the transversely-extending port 156 so that the valve shaft 120 may block fluid communication of the transversely-extending port 156 with the valve well passage 152 when the valve shaft 120 is in the off position illustrated in FIG. 2A. Thus, in contrast with prior art valve shafts, a valve shaft 120 formed in accordance with various principles of the present disclosure may be of a more limited length and shorter than prior art valve shafts. With a generally different configuration than prior art valve shafts, a valve shaft 120 formed in accordance with various principles of the present disclosure may be considered or referred to as a stopper, blocker, plunger, plug, etc. The valve shaft 120 is formed of a material capable of sealing with respect to the wall of the valve well 150 defining the valve well passage 152, and particularly with respect to the transversely-extending port 156 of the valve well 150. For instance, the valve shaft 120 may be formed of an elastomeric material, such as a thermoplastic elastomer, or metal (e.g., stainless steel) allowing for sufficiently tight tolerances to achieve the desired sealing. The exterior surface of the valve shaft 120 may have a substantially continuous or smooth surface and may be sized, shaped, configured, and/or dimensioned to allow shifting of the valve shaft 120 along the actuation axis A while also providing sealing with respect to the transversely-extending port 156 and the axially-extending port 154. In some embodiments, the valve shaft 120 includes circumferential seal elements 122a, 122b circumferentially-extending radially-projecting seal elements extend around the circumference of the valve shaft 120, as illustrated in FIG. 6A and FIG. 6B.

In accordance with various principles of the present disclosure, and as may be appreciated upon comparison of the relative positions of the valve shaft 120 and user-engagement element 130 of the example of an embodiment of a valve assembly 100 illustrated in FIG. 2A with FIG. 2B, actuation of the actuatable member 110 causes movements of valve shaft 120 and user-engagement element 130 in opposite directions. Specifically, shifting of the user-engagement element 130 from a neutral proximal position (as in FIG. 2A) distally into a more a distal position (toward the distal end 103 of the valve assembly 100, as illustrated in FIG. 2B) causes the valve shaft 120 to move proximally. Thus, in the example of an embodiment of a valve assembly 100 illustrated in FIG. 2A and FIG. 2B, when the actuatable member 110 is in a generally neutral position, the valve assembly 100 is in an off configuration with the valve shaft 120 in a position blocking fluid communication between the axially-extending port 154 and the transversely-extending port 156. The actuatable member 110 of the illustrated example of an embodiment is shifted distally to shift the valve assembly 100 into an on configuration, in which the valve shaft 120 is moved out of the flow path between the axially-extending port 154 and the transversely-extending port 156 (via the valve well passage 152). As may be appreciated, the valve shaft 120 must be moved proximally in order to allow fluid flow between the axially-extending port 154, located at a distal end 153 of the valve well 150, and the transversely-extending port 156, which is located proximal to the axially-extending port 154 (closer to the proximal end 153 of the valve well 150). Typically, the actuation direction of an actuatable member 110 is distal, i.e., a movement depressing the user-engagement element 130 toward the distal end 103 of the valve assembly 100. Therefore, the actuatable member 110 is configured so that distal movement of the user-engagement element 130 causes proximal movement of the valve shaft 120 to shift the valve shaft 120 from an off (or closed) position to an on (or open) position. In accordance with various principles of the present disclosure, the linkage assembly coupling the user-engagement element 130 and the valve shaft 120 is configured to effect the opposite relative motions described above. Furthermore, in accordance with various principles of the present disclosure, a biasing assembly made up of the linkage assembly and a biasing element, may be configured maintain the valve assembly 100 in an off configuration, with active actuation by the user (i.e., intentional input of an actuation force) to the user-engagement element 130 shifting the valve assembly 100 to an open configuration as desired by the user.

In the example of an embodiment of an actuatable member 110 illustrated in FIG. 2A and FIG. 2B, the linkage assembly 140 includes a plurality of linkages 142a, 142b, 142c, 142d, pivotably coupled with respect to one another as well as with respect to the user-engagement element 130 and the valve shaft 120. For instance, the proximal ends 141a, 141b of the proximal linkages 142a, 142b, respectively, are pivotably coupled with respect to the user-engagement element 130, and the distal ends 143c, 143d of the distal linkages 142c, 142d, respectively, are pivotably coupled with respect to the valve shaft 120. The pivotable connections may be any suitable configuration known to those of ordinary skill in the art. An example of an embodiment of a pivotable connection is a pivot pin 144 at an end of a linkage 142a, 142b pivotably mounted in a suitable pivot bracket 134 mounted on the user-engagement element 130 and/or a pivot pin 144 at an end of a linkage 142c, 142d pivotably mounted in a suitable pivot bracket 124 mounted on the valve shaft 120. A similar element, such as another pivot pin 144, may guide the linkages 142a, 142b, 142c, 142d with respect to the guide path 162, such as by sliding along a guide slot 164 formed with respect to the guide path 162. However, the present disclosure need not be limited in this regard.

In the example of an embodiment of an actuatable member 110 illustrated in FIG. 2A and FIG. 2B, the ends of the linkages not pivotably coupled to the valve shaft 120 or the user-engagement element 130 are guided by a guide path 162. More particularly, the distal ends 143a, 143b of the proximal linkages 142a, 142b, respectively, and the proximal ends 141c, 141d of the distal linkages 142c, 142d, respectively, are guided by the guide path 162. In the example of an embodiment illustrated in FIG. 2A and FIG. 2B, the guide path 162 is mounted with respect to the collar 160. The linkages 142a, 142b, 142c, 142d, may be considered to be coupled with respect to as well as movable with respect to one another via the guide path 162. A skirt 136 extending distally from the user-engagement surface 132 of the user-engagement element 130 may extend circumferentially around the linkage assembly 140 as well as around the collar 160 and the guide path 162 mounted thereto when the actuatable member 110 is shifted distally with respect to the collar 160.

As the actuatable member 110 is shifted distally, the ends 143a, 143b, 141c, 141d of the linkages 142a, 142b, 142c, 142d are guided, along a guide axis G along the guide path 162, away from a rest position along a medial region of the actuatable member 110 (e.g., spaced inwardly from the skirt 136 of the user-engagement element 130, as illustrated in FIG. 2A) and in a direction laterally/radially outwardly, closer to the ends 161, 163 of the guide path 162 (as illustrated in FIG. 2B). The actuation axis A lies along a medial region of the valve assembly 100, such as along a central axis thereof, while the guide axis G extends transverse to the actuation axis A. The linkage assembly 140 thereby moves from an extended configuration (extending closer to the actuation axis A than to the ends 161, 163 of the guide axis G, as illustrated in FIG. 2A) to a contracted configuration (extending closer to the ends 161, 163 of the guide axis G than to the actuation axis A, as illustrated in FIG. 2B). It will be appreciated that reference may be made interchangeably herein to a contracted, collapsed, compressed, etc., configuration without intent to limit. As the configuration of the linkage assembly 140 shifts from the extended configuration to the contracted configuration, the valve shaft 120 is moved closer to the user-engagement element 130, and out of a fluid communication path between the axially-extending port 154 and the transversely-extending port 156 of the valve well 150.

In some situations, it is desirable for the valve assembly 100 to be in an off configuration when in a neutral position (when an actuating force is not applied to the actuatable member 110). Such situations include when a valve assembly 100 such as described herein is configured for use with an endoscope 1000 and is coupled to a suction source which is continuously running. It is generally desirable to limit suction applied by the valve assembly 100 to instances when suction is desired, and to limit, and preferably eliminate, suction force to the valve-well-suction-application port 154 when suction is not desired. For instance, in certain endoscopic procedures, it is desirable to maintain an anatomical site insufflated to improve visualization of the target site of the procedure, and/or to irrigate a target site, such as by supplying fluid to the target site. Suction may be limited to reducing the supplied fluid in certain instances, and/or to remove other materials (e.g., biological materials) from the target site. In such situations, the valve assembly 100 may be biased into the neutral off configuration so that the actuatable member 110, including the valve shaft 120 and the linkage assembly 140, are returned to an off configuration upon removal of an actuating force which had been applied to move the valve assembly 100 to an actuated, on configuration.

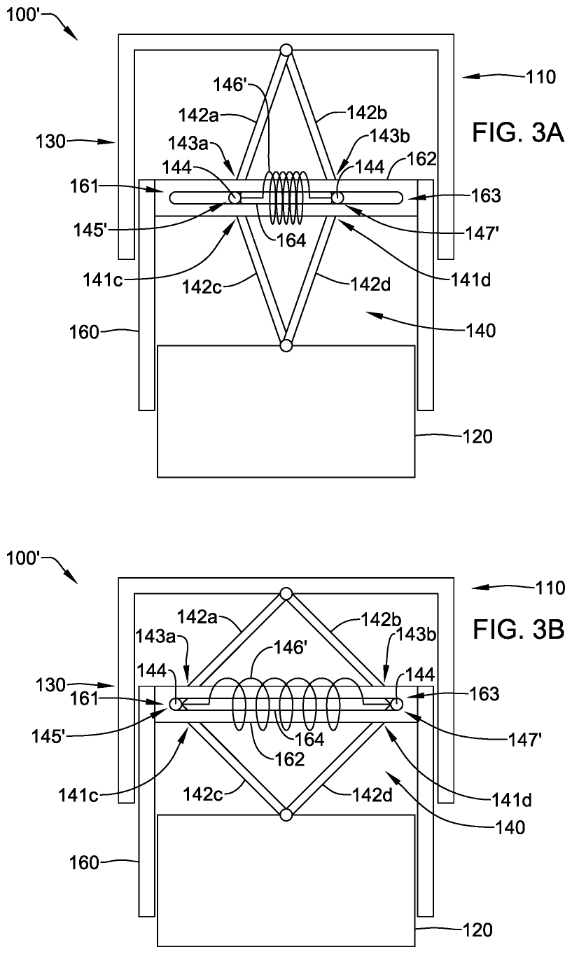

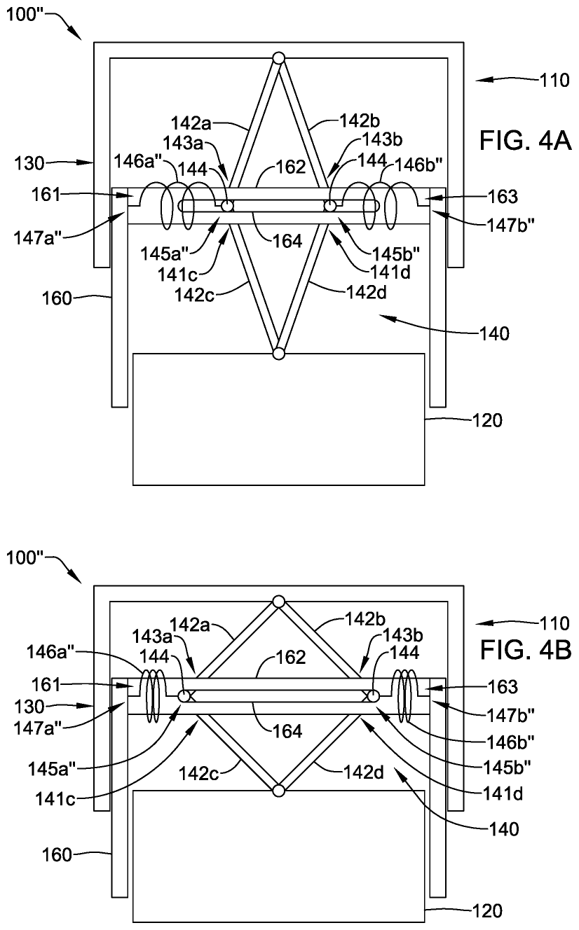

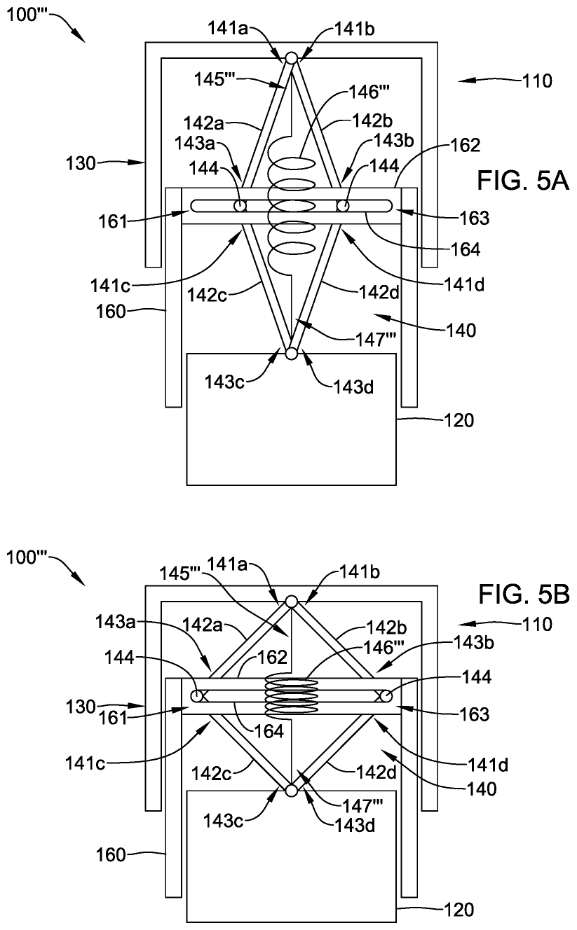

In the example of an embodiment illustrated in FIG. 2A and FIG. 2B, a biasing element may be provided to return the linkage assembly 140 to a neutral extended configuration from the contracted configuration. The present disclosure encompasses various configurations of biasing elements and arrangement thereof with respect to the linkage assembly 140, the combination of the biasing element and linkage assembly in some aspects being considered a biasing assembly, system, mechanism, etc. Examples of embodiments of such configurations are illustrated in FIG. 3A, FIG. 3B, FIG. 4A, FIG. 4B, FIG. 5A, and FIG. 5B, in which a biasing element is positioned with respect to the linkage assembly 140 to return the linkage assembly 140, and thus the valve assembly 100, to a neutral configuration (such as illustrated in FIG. 3A, FIG. 4A, and FIG. 5A) from an actuated configuration (such as illustrated in FIG. 3B, FIG. 4B, and FIG. 5B). The biasing element may be a tension spring or a compression spring. It will be appreciated that elements of the valve assemblies 100′, 100″, 100′″ illustrated in FIG. 3A, FIG. 3B, FIG. 4A, FIG. 4B, FIG. 5A, and FIG. 5B which are similar to elements of the example of an embodiment of a valve assembly 100 illustrated in FIG. 2A and FIG. 2B are indicated with the same reference characters, and reference is made to the descriptions thereof provided above for the sake of brevity and without intent to limit. Furthermore, although the biasing elements may differ among the various examples of embodiments illustrated in FIG. 3A, FIG. 3B, FIG. 4A, FIG. 4B, FIG. 5A, and FIG. 5B, and may affect the relative positions of the linkages 142a, 142b, 142c, 142d, the linkage assemblies 140 across the examples of embodiments illustrated in FIG. 3A, FIG. 3B, FIG. 4A, FIG. 4B, FIG. 5A, and FIG. 5B may otherwise be similar and are thus referenced with the same reference characters, and reference is made to descriptions thereof provided above, without intent to limit. It is noted that slight differences in the arrangements of the ends of the linkages 142a, 142b, 142c, 142d among FIG. 2A, FIG. 2B, FIG. 3A, FIG. 3B, FIG. 4A, FIG. 4B, FIG. 5A, and FIG. 5B are illustrated and do not affect operation of the linkage assemblies 140 described herein or the general principles of the present disclosure.

In the example of an embodiment of a valve assembly 100′ illustrated in FIG. 3A and FIG. 3B, a biasing element in the form of a tension spring 146′ extending along the guide axis G is provided with respect to the linkage assembly 140. One end 145′ of the spring 146′ is coupled to the ends 143a, 141c of the linkages 142b, 142d which move towards the end 161 of the guide path 162 as the actuatable member 110 is shifted to the on position thereof. The other end 147′ of the spring 146′ is coupled to the ends 143b, 141d of the linkages 142b, 142d which move towards the end 163 of the guide path 162 (opposite the end 161) as the actuatable member 110 is shifted to the on position. As the user-engagement element 130 is shifted distally to shift the valve assembly from the configuration of FIG. 3A to the configuration of FIG. 3B, tension is applied to the spring 146′. Such tension in the spring 146′ causes the linkage assembly 140 to return to the configuration illustrated in FIG. 3A once an actuation force (in the distal direction towards the distal end 103 of the valve assembly 100′) is no longer applied to the actuatable member 110.

In the example of an embodiment of a valve assembly 100″ illustrated in FIG. 4A and FIG. 4B, a biasing element in the form of a pair of compression springs 146a″, 146b″ extending along the guide axis G is provided with respect to the linkage assembly 140. One end 145a″ of the spring 146a″ is coupled to the ends 143a, 141c of the linkages 142a, 142c which move towards the end 161 of the guide path 162 as the actuatable member 110 is shifted to the on position thereof. The other end 147a″ of the spring 146a″ is coupled to the end 161 of the guide path 162. Similarly, one end 145b″ of the spring 146b″ is coupled to the ends 143b, 141d of the linkages 142b, 142d which move towards the end 163 of the guide path 162 (opposite the end 161) as the actuatable member 110 is shifted to the on position. The other end 147b″ of the spring 146b″ is coupled to the end 163 of the guide path 162. As the user-engagement element 130 is shifted distally to shift the valve assembly 100″ from the configuration of FIG. 4A to the configuration of FIG. 4B, the springs 146a″, 146b″ are compressed. The potential energy created by compressing the spring 146a″, 146b″ allows the springs 146a″, 146b″ to return the linkage assembly 140 to the configuration illustrated in FIG. 4A once an actuation force (in the distal direction towards the distal end 103 of the valve assembly 100″) is no longer applied to the actuatable member 110.

In the example of an embodiment of a valve assembly 100′″ illustrated in FIG. 5A and FIG. 5B, a biasing element in the form of a compression spring 146′″ extending along the actuation axis A is provided with respect to the linkage assembly 140. A proximal end 145′″ of the spring 146′″ is coupled to the proximal ends 141a, 141b of the proximal linkages 142a, 142b coupled to the user-engagement element 130. The distal end 147′″ of the spring 146′″ is coupled to the distal ends 143c, 143d of the distal linkages 142c, 142d coupled to the valve shaft 120. As the user-engagement element 130 is shifted distally to shift the valve assembly 100′″ from the configuration of FIG. 5A to the configuration of FIG. 5B, the spring 146′″ is compressed. The potential energy created by compressing the spring 146′″ allows the spring 146′″ to return the linkage assembly 140 to the configuration illustrated in FIG. 5A once an actuation force (in the distal direction towards the distal end 103 of the valve assembly 100″) is no longer applied to the actuatable member 110.

It will be appreciated that the present disclosure need not be limited to the illustrated and above-described configurations of biasing elements and/or linkage assemblies, or to the illustrated and above-described configurations of biasing elements with respect to linkage assemblies. For instance, instead of a biasing assembly having a biasing element with (and separate from) a linkage assembly, a biasing assembly of an actuatable member of a valve assembly formed in accordance with various principles of the present disclosure may have a linkage assembly which itself is made of resilient, springy, elastic, etc., material which returns the actuatable member to a neutral position (e.g., spring steel, music wire, Nitinol wire, etc.). As such, the biasing assembly may be considered to be formed of a linkage assembly which also functions as a biasing element of the biasing assembly. For instance, the linkage assembly may include one or more linkages, each linkage having a first end coupled (e.g., pivotably) with a user-engagement element and a second end coupled (e.g., pivotably) with a valve shaft, and each linkage being capable of biasing apart the user-engagement element and the valve shaft. Additionally or alternatively, instead of two or more linkages extending between the user-engagement element and valve shaft of the actuatable member, with intermediate ends guided along a guide path, such as in the examples of embodiments illustrated in FIG. 3A, FIG. 3B, FIG. 4A, FIG. 4B, FIG. 5A, and FIG. 5B, a single linkage may extend between the user-engagement element and the valve shaft, with a proximal end of the single linkage coupled to the user-engagement element and the distal end of the single linkage coupled to the valve shaft. One or more such single linkages may be provided.

An example of an embodiment of a valve assembly 200 with a linkage assembly 240 including a pair of single linkages (i.e., a first linkage and a second linkage together forming a pair of linkages forming a linkage assembly), each linkage having a proximal end coupled to a user-engagement element of an actuatable member and a distal end coupled to a valve shaft of the actuatable member, is illustrated in FIG. 6A and FIG. 6B. It will be appreciated that elements of the valve assembly 200 illustrated in FIG. 6A, and FIG. 6B which are similar to elements of the example of an embodiment of a valve assembly 100 illustrated in FIG. 2A and FIG. 2B are indicated with the same reference characters, and reference is made to the descriptions thereof provided above for the sake of brevity and without intent to limit. In the example of an embodiment of a valve assembly 200 illustrated in FIG. 6A and FIG. 6B, a pair of linkages 242a, 242b are provided between the user-engagement element 130 and the valve shaft 120, each linkage 242a, 242b having a respective proximal end 241a, 241b coupled with the user-engagement element 130, and a distal end 243a, 243b coupled with the valve shaft 120. An intermediate region 245a, 245b of the respective linkages 242a, 242b is engaged with and guided by the guide path 162. The ends 241a, 241b, 243a, 243b of linkages 242a, 242b may be pivotably coupled, respectively, with user-engagement element 130 and the valve shaft 120. As with the examples of embodiments illustrated in FIG. 3A, FIG. 3B, FIG. 4A, FIG. 4B, FIG. 5A, and FIG. 5B, the pivotable connections may be any suitable configuration known to those of ordinary skill in the art. An example of an embodiment of a pivotable connection is a pivot pin 244 at an end 241a, 241b, 243a, 243b of a linkage 242a, 242b pivotably mounted in a suitable pivot bracket 134, 124 mounted on the user-engagement element 130 and the valve shaft 120. However, the present disclosure is not limited in this regard. Similarly, the intermediate regions 245a, 245b of the respective linkages 242a, 242b may include an element such as a pivot pin 244 guided with respect to a guide slot 164 formed with respect to a guide path 162.

As the user-engagement element 130 of the actuatable member 110 of the example of an embodiment of a valve assembly 200 illustrated in FIG. 6A and FIG. 6B is shifted distally, the linkages 242a, 242b shift from a generally elongated extended configuration, as illustrated in FIG. 6A, to a compressed configuration, as illustrated in FIG. 6B. For instance, the linkages 242a, 242b flex into the compressed configuration from the extended configuration. In embodiments in which the linkages 242a, 242b include a guide pin (e.g., pivot pin 144), the guide path 162 may guide the movement of the intermediate regions 245a, 245b of the respective linkages 242a, 242b along the guide axis G and laterally away from the actuation axis A. Such compression of the linkages 242a, 242b upon distal movement of the user-engagement element 130 draws the valve shaft 120 proximally. As with the above-described examples of embodiments illustrated in FIG. 2A, FIG. 2B, FIG. 3A, FIG. 3B, FIG. 4A, FIG. 4B, FIG. 5A, and FIG. 5B, proximal movement of the valve shaft 120 allows the axially-extending port 154 and the transversely-extending port 156 of the valve well 150 to be in fluid communication, shifting the valve assembly 200 into an on configuration. The resilience, elasticity, springiness, etc., of the linkages 242a, 242b may bias the user-engagement element 130 proximally upon withdrawal of an actuation force (in the distal direction towards the distal end 103 of the valve assembly 200) thereto, to return the actuatable member 110 to the off position and the valve assembly 200 to the off configuration illustrated in FIG. 6A.

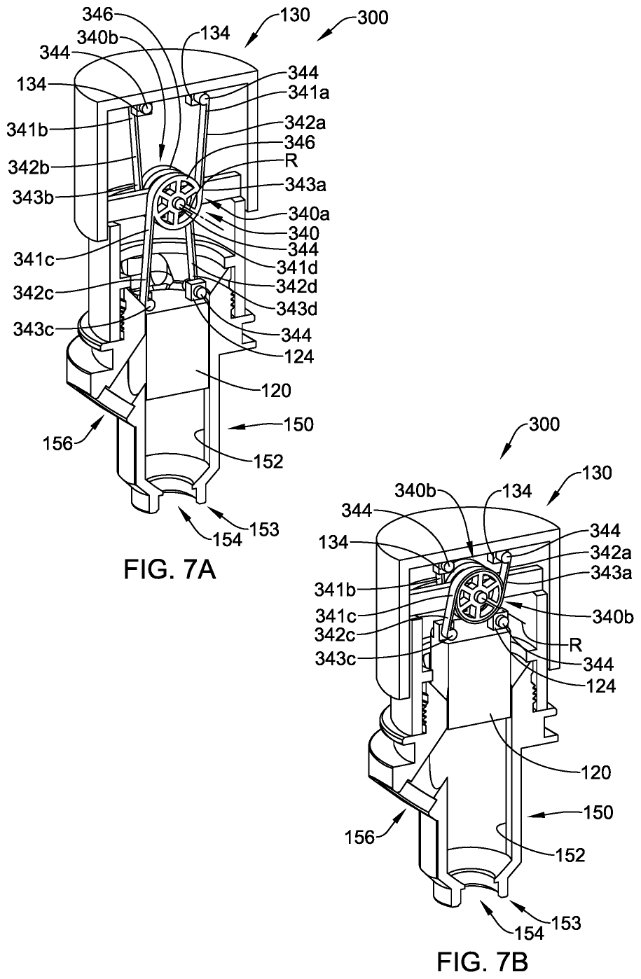

Instead of generally linear biasing members and/or linkages forming a biasing assembly (such as in the example of an embodiment illustrated in FIG. 6A and FIG. 6B), a biasing member using and/or applying torsional forces may be used to bias the user-engagement element 130 with respect to the valve shaft 120 of a valve assembly formed in accordance with various principles of the present disclosure. For instance, the example of an embodiment of a valve assembly 300 illustrated in FIG. 7A and FIG. 7B has a biasing assembly 340 including a biasing structure configured and adapted to perform substantially as a torsional spring (e.g., storing and then releasing non-linear rotational rather than linear energy). Similar to the above-described valve assemblies 100, 100′, 100″, 100′″, 200, the biasing assembly 340 maintains the user-engagement element 130 and the valve shaft 120 spaced apart from each other to maintain the valve assembly 300 in a closed, off configuration. Also as with the above-described examples of embodiments illustrated in FIG. 2A, FIG. 2B, FIG. 3A, FIG. 3B, FIG. 4A, FIG. 4B, FIG. 5A, FIG. 5B, FIG. 6A, and FIG. 6B, application of an actuation force to the user-engagement element 130 effects proximal movement of the valve shaft 120, which allows the axially-extending port 154 and the transversely-extending port 156 of the valve well 150 to be in fluid communication, shifting the valve assembly 200 into an on configuration. Once the actuation force is no longer applied to the user-engagement element 130, the biasing assembly 340 (specifically, forces stored therein/thereby) return the user-engagement element 130 and the valve shaft 120 to a spaced-apart configuration, shifting the valve assembly 300 from the open, on configuration back to the closed, off configuration. The closed, off configuration and the open, on configuration of the valve assembly 300 may be generally similar to the closed, off configuration and the open, on configuration of the above-described valve assemblies 100, 100′, 100″, 100′″, 200, reference being made to the above-descriptions of such configurations as applying to the valve assembly 300 illustrated in in FIG. 7A and FIG. 7B for the sake of brevity, and without intent to limit. It will be appreciated that elements of the valve assembly 300 illustrated in FIG. 7A, and FIG. 7B which are similar to elements of the example of an embodiment of a valve assembly 100 illustrated in FIG. 2A, FIG. 2B, FIG. 3A, FIG. 3B, FIG. 4A, FIG. 4B, FIG. 5A, FIG. 5B, FIG. 6A, and FIG. 6B are indicated with the same reference characters and/or reference characters increased by a factor of 100, and reference is made to the descriptions thereof provided above for the sake of brevity and without intent to limit.

In the example of an embodiment of a biasing element 340 illustrated in FIG. 7A and FIG. 7B, the biasing assembly 340 includes one or more biasing assembly units 340a, 340b, each having a proximal arm or linkage 342a, 342b and a distal arm or linkage 342c, 342d, the linkages 342a, 342b, 342c, 342d being coupled to a rotational element 346. It will be appreciated that terms such as arm or linkage may be used interchangeably herein with reference to the linkages 342a, 342b, 342c, 342d without intent to limit. It will further be appreciated that the rotational element 346 may be a wheel, hub, etc., about which the linkages 342a, 342b, 342c, 342d may be wrapped to store rotational/torsional energy in the biasing assembly 340. The biasing assembly 340 may be formed of a metal such as stainless steel, spring steel, nitinol (or other shape memory material/alloy), etc., or a plastic.

The linkages 342a, 342b, 342c, 342d are coupled with the user-engagement element 130 and the valve shaft 120 as well as with the rotational element 346. In some aspects, the linkages 342a, 342b, 342c, 342d may be considered to link the user-engagement element 130 and the valve shaft 120 of the valve assembly 300. In some aspects, the proximal ends 341a, 341b of the proximal linkages 342a, 342b, respectively, are pivotably coupled with respect to the user-engagement element 130, and the distal ends 343c, 343d of the distal linkages 342c, 342d, respectively, are pivotably coupled with respect to the valve shaft 120. The pivotable connections may be any suitable configuration known to those of ordinary skill in the art. An example of an embodiment of a pivotable connection is a pivot pin 344 at an end of a linkage 342a, 342b pivotably mounted in a suitable pivot bracket 134 mounted on the user-engagement element 130 and/or a pivot pin 344 at an end of a linkage 342c, 342d pivotably mounted in a suitable pivot bracket 124 mounted on the valve shaft 120. However, the present disclosure is not limited in this regard. In some aspects, the distal ends 343a, 343b of the proximal linkages 342a, 342b, respectively, and the proximal ends 341c, 341d of the distal linkages 342c, 342d, respectively, are coupled with respect to respective rotational elements 346 to facilitate bending or flexing of the linkages 342a, 342b, 342c, 342d to wrap around the rotational element 346 when the user-engagement element 130 and valve shaft 120 are moved closer together/towards each other as described in further detail below. In some aspects, the coupling of the linkages 342a, 342b, 342c, 342d with the rotational element 346 may allow a degree of pivoting of the linkages 342a, 342b, 342c, 342d with respect to the rotational element 346 less than the pivoting resulting from the above-described pivoting about pivot pins, and thus is not about a pivot pin but a more fixed yet flexible/non-rigid connection which still allows pivoting.

The rotational element 346 is rotatably mounted with respect to the valve assembly 300 (e.g., via a pin 344, axle, etc.) so that upon applying an actuation force to the user-engagement element 130, the linkages 342a, 342b, 342c, 342d are moved closer together/towards each other and caused to wrap around the rotational element 344, the rotational element 346 is thereby caused to rotate about rotation axis R, and the user-engagement element 130 and the valve shaft 120 are brought together to shift the valve assembly 300 into an open, on configuration. As may be appreciated, the wrapping of the linkages 342a, 342b, 342c, 342d around the rotational element 346 stores rotational (torsion spring) energy in the linkages 342a, 342b, 342c, 342d. When actuation force is removed from the user-engagement element 130, the stored force in the linkages 342a, 342b, 342c, 342d causes the rotational element 344 to rotate in the opposite direction with the linkages 342a, 342b, 342c, 342d unwrapping or straightening to move the user-engagement element 130 and valve shaft 120 apart to shift the valve assembly 300 into the closed, off configuration.

In some aspects, for torsional forces to be effectuated by the rotational element 346, the ends 341a, 341b, 343c, of the linkages 342a, 342b, 342c, 342d coupled with the user-engagement element 130 and valve shaft 120 are mounted on generally diametrically opposed sides, offset from the rotational axis R of the rotational element 346. As may be appreciated, such positions maximize the moment arm of the linkages 342a, 342b, 342c, 342d with respect to the rotational element 346. The greater the distance of the pivot connection of the linkages 342a, 342b, 342c, 342d to the user-engagement element 130 and valve shaft 120 from the rotational axis R, the greater the moment arm which causes rotation of the rotational element 346 and wrapping of the linkages 342a, 342b, 342c, 342d therearound. However, it will be appreciated that such distance may be limited by the dimensions of the space within the valve assembly 300, such as inner diameter of the passage 152 within the valve well 150. Alternatively or additionally, the ends 343a, 343b, 341c, 341d of the linkages 342a, 342b, 342c, 342d, respectively, which are coupled with a respective rotational element 346 may be attached tangentially to the outer perimeter of the rotational element 346. In other words, the linkages 342a, 342b, 342c, 342d may be considered flat, ribbon-like spring arms with an end tangent to and coupled with the rotational element 346. Such configuration may facilitate wrapping of the linkages 342a, 342b, 342c, 342d around the rotational element 346 and storing torsional forces therein. As may be appreciated, the widths of the linkages 342a, 342b, 342c, 342d may be determined based on the space within the valve assembly 300, such as inner diameter of the passage 152 within the valve well 150 Alternatively or additionally, it will be appreciated that the diameter of the rotational element 346 may affect the amount of energy stored by the biasing assembly 340 and/or the ease at which the valve assembly 300 is shifted between on and off configurations. Although a larger diameter rotational element 346 generally would provide a better mechanical advantage, as with other components of the biasing assembly 340, the dimensions of the rotational element 346 may be limited by the space within the valve assembly 300, such as inner diameter of the passage 152 within the valve well 150 and/or the internal distance between the user-engagement element 130 and the valve shaft 120.

In situations in which a valve assembly as described herein is fluidly coupled with a suction source which is continuously on during use of the valve assembly, it may be desirable to vent or bleed vacuum pressure generated within the valve assembly from the suction source when the valve assembly is in the off configuration. In accordance with various principles of the present disclosure, one or more bleed passages may be defined in the valve assembly to bleed/vent ambient air to the suction source. As may be appreciated, the positions and configurations of such bleed passages will depend on the location of the valve shaft with respect to the valve well port in fluid communication with the suction source. For instance, if the suction source is fluidly-coupled with the transversely-extending port of the valve well, then a bleed passage may be formed along a proximal end of the valve shaft to fluidly communicate the transversely-extending port with ambient air while the valve shaft is in an off position. The longitudinal extent of such bleed passage may be limited so that the flow path between the transversely-extending port and the axially-extending port is sealed from ambient air when the valve shaft is in an on position, so that suction applied to the axially-extending port from the transversely-extending port is not lost to ambient. If the axially-extending port of the valve well is fluidly coupled with a suction source, then a bleed passage defined with respect to the valve shaft and the valve well may extend axially with respect thereto when the valve shaft is in an off position, but would be blocked when the valve shaft is shifted to an on position. For example, a bleed passage may extend axially through the valve shaft 120. When the valve shaft 120 is moved upwards, such a passage could then be blocked by a feature on the bottom of guide path 162 to cut off the bleed passage when the suction source is in fluid communication with the suction application port.

Various further benefits of the various aspects, features, components, and structures of a valve shaft and associated seal members, as well as valve assemblies and endoscopes such as described above, in addition to those discussed above, may be appreciated by those of ordinary skill in the art.

It is to be understood by one of ordinary skill in the art that the present discussion is a description of illustrative examples of embodiments only, and is not intended as limiting the broader aspects of the present disclosure. It will be appreciated that principles of the present disclosure may be applied to various medical devices, instruments, tools, etc., such, without limitation, a variety of medical devices, instruments, tools, etc., for accessing anatomical sites and applying suction and/or irrigation thereto, including, for example, endoscopes, gastroscopes, duodenoscopes, catheters, ureteroscopes, bronchoscopes, colonoscopes, arthroscopes, cystoscopes, hysteroscopes, and the like, having integrated features for suction and/or irrigation of anatomical sites. Moreover, principles of the present disclosure may be applied to reusable or single-use devices, instruments, tools, etc.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. For instance, various elements and components of a valve assembly described herein may be coupled or engaged directly or indirectly with one another, regardless of how such connections are depicted in the drawings. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An actuatable member for a valve assembly of a medical instrument, said actuatable member having a proximal end and a distal end and comprising:
a user-engagement element along the proximal end thereof; and
a shaft along the distal end thereof and movable with respect to said user-engagement element;
wherein:
said actuatable member is extendable through a valve well passage of a valve well of the medical valve assembly and shiftable with respect thereto, along an actuation axis, between a first position and a second position; and
distal movement of said actuatable member toward the distal end of said valve well causes movement of said valve shaft toward said user-engagement element.

2. The actuatable member of claim 1, further comprising a biasing assembly coupling said user-engagement element and said valve shaft.

3. The actuatable member of claim 2, wherein said biasing assembly is configured to shift between an extended configuration in which said user-engagement element and said valve shaft are spaced apart from each other a first distance, and a contracted configuration in which said user-engagement element and said valve shaft are spaced apart from each other a second distance shorter than the first distance.

4. The actuatable member of claim 3, wherein said biasing assembly comprises a linkage assembly comprising a plurality of linkages pivotable coupled together between said user-engagement element and said valve shaft.

5. The actuatable member of claim 4, wherein said plurality of linkages comprise:
proximal linkages having proximal ends and distal ends; and
distal linkages having proximal ends and distal ends;
wherein:
the proximal ends of said proximal linkages are coupled to said user-engagement element;
the distal ends of said distal linkages are coupled to said valve shaft; and
the distal ends of said proximal linkages and the proximal ends of said distal linkages are pivotably coupled with respect to one another.

6. The actuatable member of claim 5, further comprising a guide path positioned with respect to said linkage assembly to guide a portion of said one or more linkages to move said linkages between the extended configuration and the compressed configuration.

7. The actuatable member of claim 6, wherein the distal ends of said proximal linkages and the proximal ends of said distal linkages are guided with respect to said guide path to move along a guide axis transverse to the actuation axis to shift the linkage assembly between the expanded configuration and the contracted configuration.

8. The actuatable member of claim 3, wherein said linkage assembly comprises at least one linkage having a proximal end coupled to said user-engagement element and a distal end coupled to said valve shaft.

9. The actuatable member of claim 8, wherein:
said at least one linkage is formed of a resilient material; and
distal movement of said user-engagement element toward the distal end of said valve well causes said at least one linkage to flex and to shift said valve shaft toward the proximal end of said valve well.

10. The actuatable member of claim 1, wherein said biasing assembly comprises a linkage assembly having two or more linkage arms, and a rotational element about said linkage arms wrap as said user-engagement element and said valve shaft are moved towards each other.

11. The actuatable member of claim 1, wherein said user-engagement element is biased away from the distal end of said valve well.

12. The actuatable member of claim 1, wherein:
an axially-extending port is in fluid communication with the valve well passage along the actuation axis;
a transversely-extending port is in fluid communication with the valve well passage in a direction transverse to the actuation axis; and
distal movement of said user-engagement element shifts said valve shaft from a position blocking fluid communication between the axially-extending port and the transversely-extending port via the valve well passage, to a position allowing fluid communication between the axially-extending port and the transversely-extending port via the valve well passage.

13. An actuatable member for a medical valve assembly, said actuatable member having a proximal end and a distal end and comprising:
a valve shaft extendable along an actuation axis within a valve well passage of a medical valve assembly between an off position in which said valve shaft blocks fluid communication between an axially-extending port and a transversely-extending port in the valve well, and an on position in which said valve shaft is moved out of the flow path between the axially-extending port and the transversely-extending port to allow fluid communication therebetween.

14. The actuatable member of claim 13, further comprising a user-engagement element coupled to said valve shaft and movable distally to shift said valve shaft from the valve shaft off position to the valve shaft on position.

15. The actuatable member of claim 14, wherein:
when said valve shaft is in the off position, said valve shaft is positioned to block a flow path between the axially-extending port and the transversely-extending port of the valve well; and
when said valve shaft is in the on position, said valve shaft is positioned proximal to the transversely-extending port and out of the flow path between the axially-extending port and the transversely-extending port.

16. The actuatable member of claim 14, further comprising a biasing assembly coupling said user-engagement element and said valve shaft.

17. The actuatable member of claim 16, wherein said biasing assembly comprises a linkage assembly including one or more linkages configured to shift between an extended configuration in which said user-engagement element and said valve shaft are spaced apart from each other a first distance, and a contracted configuration in which said user-engagement element and said valve shaft are spaced apart from each other a second distance shorter than the first distance.

18. The actuatable member of claim 17, further comprising a guide path positioned with respect to said linkage assembly to guide a portion of said one or more linkages to move said linkages between the extended configuration and the contracted configuration.

19.

20. A method of applying suction via an endoscope, said method comprising shifting a valve shaft of an actuatable member along an actuation axis and within a valve well passage between an off position in which the valve shaft blocks fluid communication between an axially-extending port and a transversely-extending port via the valve well passage, and an on position in which the valve shaft is moved out of the flow path between the axially-extending port and the transversely-extending port via the valve well passage to allow fluid communication therebetween.

21. The method of claim 19, further comprising:
distally shifting a user-engagement element of the actuatable member in a direction towards the control handle of the endoscope to cause the valve shaft to shift proximally toward the user-engagement element and out of the flow path between the axially-extending port and the transversely-extending port via the valve well passage.
